(19) United States
(12) Patent Application Publication
(10) Pub. No.: US 2012/0276152 A1
(43) Pub. Date: Nov. 1, 2012

(54) SYSTEMS AND METHODS OF USING ZINC-CHELATOR TO TREAT MYOCARDIAL INFARCTION

(76) Inventors: Syed Hossainy, Hayward, CA (US); John Stankus, Campbell, CA (US); Mikael Trollsas, San Jose, CA (US); Dariush Davalian, San Jose, CA (US)

(21) Appl. No.: 13/366,841

(22) Filed: Feb. 6, 2012

Related U.S. Application Data
(63) Continuation-in-part of application No. 13/098,055, filed on Apr. 29, 2011.

(51) Int. Cl. A61K 9/00; A61K 31/351; A61K 31/4412; A61K 31/44; A61K 38/02; A61K 31/734; A61K 31/045; A61K 33/26; A61K 31/04; A61K 31/519; A61K 31/4985; A61K 31/352; A61K 38/22; A61K 31/198; A61K 33/00; A61K 31/502; A61K 47/30; A61P 9/10; A61K 31/16

(52) U.S. Cl. 424/400; 514/575; 514/625; 514/460; 514/345; 514/348; 514/1.1; 514/54; 514/724; 424/608; 514/727; 514/252.16; 514/250; 514/454; 514/12.4; 514/565; 424/718; 514/248; 514/772.3

(57) ABSTRACT

Methods and systems for treating an infarct by delivery of zinc chelator to modulate tissue.

### CROSS REFERENCE TO RELATED APPLICATIONS

This application is a continuation-in-part of co-pending, U.S. patent application Ser. No. 13/098,055, filed Apr. 29, 2011, the disclosure of which is hereby incorporated by reference in its entirety.

### TECHNICAL FIELD

The subject matter relates to methods and compositions to in situ modulate mechanical properties of an infarct.

### BACKGROUND

When a patient suffers from an ischemic event in the coronary, peripheral or cerebral vasculature the blood supply to tissues and organs distal to the blockage or occlusion is significantly diminished. The resulting deprivation of oxygen increases the risk of necrosis of the tissues and organs. The infarct, or area of tissue death, from the lack of oxygen can progress to congestive heart failure if left untreated.

Some desired output variables of an infracted myocardium progressing into CHF include increasing left ventricular ejection fraction (LVEF); increasing fractional shortening; decreasing the infarct size; decreasing myocardium wall stress; increasing the wall thickness of the infarct; improving the mechanical properties of the infarct area as a function of time; improving the subject's CHF clinical classification; improving a subject's physical exercise tolerance; reducing hospitalization; and reducing the need for anti-failure medication. Additionally, important manipulated variables in an infracted myocardium progressing into CHF include cell-cell communication (autocrine and paracrine); continuous self-reinforcing apoptosis process; myocyte migration/differentiation in the hibernating area bordering the necrosed area of the infarct; progress loss of mechanical property in the infracted tissue; and myocardial collagen content.

There is a present need for methods and systems capable of modifying the mechanical or physical properties of the infracted area. Such needs can be achieved by the disclosed methods and systems.

### SUMMARY

In accordance with various aspects of the disclosed subject matter, methods, formulations, and medical devices can be used to treat an infarct and in particular modulate tissue in a positive way after an ischemic event. Alternatively, the methods, formulations, and medical devices can be useful in treating an aneurysms by administration of a zinc chelator formulation into the vasculature.

The formulation comprises a zinc chelator, a biomaterial, and a transmural transport enhancer. The transmural transport enhancer can be but is not limited to a vasodilator. A “zinc chelator” as used herein refers to a compound or molecule that can bind, ligand, or chelate zinc molecule. Some examples of vasodilators include ethanol, NO inducers such as sodium nitroprusside, nitroglycerin, sildenafil, Tadalafil, THC, atrial natriuretic peptide, adenosine, prostacyclin, nitric oxide, histamine, L-arginine, alpha blockers, ACE inhibitors, ARBs, etc.

In accordance with another aspect, an endoluminal medical device that includes formulation including zinc chelator, biomaterial, and transmural transport enhancer is provided. The formulation is disposed on the outer surface of the medical device and can in some instances be incorporated into a coating applied to the outer surface. In this regard, the zinc formulation can be delivered locally to the infarct area.

In yet another aspect, a method is disclosed for remodeling tissue. The method includes delivering a zinc chelator formulation is provided. The formulation is delivered to the myocardium where Zn++ can be essential for myocardial recovery and may be abundant in the infarct area due to matrix metalloproteinases (MMP) activity. The method also includes increasing cardioprotective effects by the introduction of Zn++ to the infracted myocardium. The mechanism of action is the zinc chelator causing a decrease in MMP activity resulting in attenuation of progression to heart failure. MMP generally decreases as a function of wall thickness. Also, MMP inhibition may be a feedback loop to help increase vessel leakiness for material uptake. MMPs are zinc dependent enzymes that degrade extracellular matrix proteins. MMPs can be inhibited by synthetic chelating molecules that strongly bind the zinc atom of the MMP active site. Some chelating groups include hydroxamates, carboxylates, and thiols.

In some embodiments, the zinc chelator in the formulation may enhance crosslinking and gelation of an infused biomaterial gel in situ in order to improve the mechanical properties of the infarct. The Zn chelator may also lower MMP activity. The increased modulus will hinder local material strength loss and gradually train the myocardium to regress into functional competency. The formulation may inhibit some MMPs, such as MMP14 and TGF-β, which can cause blood vessels to become leaky and enhance biomaterial delivery to the infarct via intracoronary delivery. In some embodiments, the chelator in the formulation may also bind to other metal ions such as Ca²⁺, K⁺, or Na⁺.

The formulation may be delivered with or without additional drugs or therapeutic agents. In some embodiments, the biologic is a protein or combination of multiple proteins such as, but not limited to, vascular endothelial growth factor (VEGF), basic fibroblast growth factor (bFGF), acidic fibroblast growth factor (aFGF), platelet-derived growth factor (PDGF), platelet-derived endothelial growth factor (PDEGF), placental derived growth factor, angiopoietin-1 (Ang-1), angiopoietin-2 (Ang-2), insulin-like growth factor 1 (IGF-1), insulin-like growth factor-2 (IGF-2), muscle derived insulin-like growth factor (mIGF), transforming growth factor-alpha (TGF-α), transforming growth factor-beta (TGF-β), hepatocyte growth factor (HGF), stem cell factor (SCF), hematopoietic growth factor or granulocyte colony-stimulating factors (G-CSF), granulocyte macrophage colony-stimulating factors (GM-CSF), nerve growth factor (NGF), growth differentiation factor-9 (GDF9), epidermal growth factor (EGF), stromal derived growth factor-1α (SDF-1α) neurotrophins, erythropoietin (EPO), thrombopoieten (TPO), myostatin (GDF-8), leukemia inhibitory factor (LIF), tumor necrosis factor-alpha (TNF-α), sonic hedgehog protein (Shh). Additionally, anti-inflammatories may be used.

### DETAILED DESCRIPTION OF EXEMPLARY EMBODIMENTS

In accordance with one embodiment of the disclosed subject matter, a formulation is provided that is useful for treating an infarct or aneurysm. The formulation comprises a zinc chelator, a biomaterial, and a transmural transport enhancer.

In one embodiment, the zinc chelator is a pendant group of a polymer. For example, the zinc chelator can include DMHP, which is represented below.

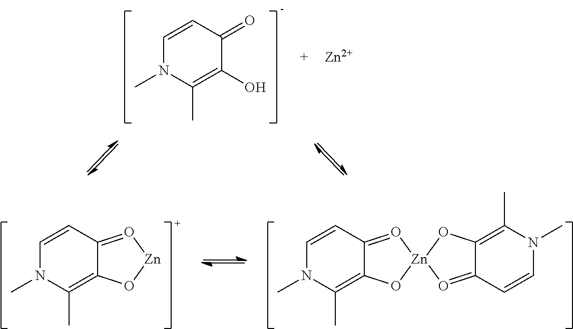

Other suitable examples include BAPTA (1,2-bis(o-aminophenyozy)ethane-N,N,N,N′,N′-tetracetic acid), as represented below.

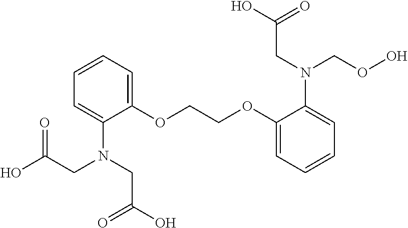

The zinc chelator can be bound to an agent. Suitable zinc binding molecules include but are not limited to: acetohydroxamic acid, N-(methyl)mercaptoacetamide, 3-Hydroxy-pyran-4-one, 1-Hydroxy-1H-pyridin-2-one, 3-Hydroxy-1-methyl-1H-pyridin-2-one, 3-Hydroxy-2-methyl-pyridin-4-one, 3-Hydroxy-1,2-dimethyl-1H-pyridin-4-one, 1-Hydroxy-1H-pyridine-2-thione, 3-Hydroxy-2-methyl-pyran-4-thione, 3-Hydroxy-1H-pyridin-2-one, 3-Hydroxy-pyran-4-thione, 3-Hydroxy-1-methyl-1H-pyridine-2-thione, and 3-Hydroxy-1,2-dimethyl-1H-pyridine-4-thione.

In one embodiment, the zinc chelator is bound to a polymer. In another embodiment, the zinc chelator is a pendant group of a biomaterial.

The biomaterial of the formulation includes [please provide biomaterials]

The transmural transport enhancer of the formulation can be for example a vasodilator. The vasodilator can enhance the mass transport of biomaterials into the infarct area by local administration to the infracted blood vessel. Suitable vasodilators include but are not limited to: ethanol and an NO inducer. Some suitable NO inducers include sodium nitroprusside, nitroglycerin, sildenafil, Tadalafil, and PETN. Other suitable vasodilators include tetrahydrocannabinol, atrial natriuretic peptide, L-arginine, NO, hydalazine, alpha blockers, ACE inhibitors and ARBs.

Formulations in accordance with the subject matter include, for example, N,N,N,N-tetrakis (2-pyridlmethyl)ethylenediamine, alginate or alginate-EDTA copolymers and nitroprusside, or N,N,N,N-tetrakis(2-pyridlmethyl)ethylenediamine, conjugated polyglutamic acid or copolymer of poly glutamic acid, alginate or alginate EDTA copolymers and nitroprusside. Yet another example of the formulation includes N,N,N,N-tetrakis(2-pyridlmethyl)ethylenediamine, conjugated alginate or alginate-EDTA copolymers, alginate or alginate EDTA copolymers and nitroprusside.

In one embodiment, the formulation is delivered to the myocardium, for example, to increase or maintain wall thickness of the infarct to attenuate heart failure. The zinc chelator can be used to enhance crosslinking and gelation of an infused biomaterial gel in situ. It is believed that such gelation would be improved mechanical properties to the infarct. The injection of a hydrogel would serve to bulk the left ventricular (LV) wall and therefore reduce wall stress. In addition, by chelating zinc from MMPs, the wall thickness will be maintained since MMPs will not degrade the LV extracellular matrix as fast. In this manner, modification of mechanical properties of the infracted area can be achieved. Such mechanical properties include: increasing LV ejection fraction, increasing fractional shortening, decreasing size of infarct, decreasing wall stress, increasing wall thickness, maintaining compliance or stiffness near that of healthy or slow change to that of failing heart.

In accordance with another aspect, an endovascular medical device including the zinc chelator formulator is provided. The endovascular medical device can achieve local delivery of the formulation at the infarct or alternatively at the site of an aneurysm such as an abdominal aortic aneurysm. In this type of administration, the endovascular medical device can be traversed through the vascular to the site of placement by entry to the left iliac artery. After deployment of the medical device, the formulation on the outer surface of the medical device can contact the vascular wall at the site of injury. The medical device can be, for example, a stent, stent-graft, or a balloon. For the purpose of illustration, the stent can be any stent design capable of carrying a formulation, such as those described in U.S. Pat. Nos. 5,902,332; 6,010,521; 6,013,069; 6,027,475; 6,036,715; 6,086,604; 6,110,142; 5,040,548; 5,061,273; 5,154,725; 5,234,002; 5,242,396; 5,350,395; 5,451,233; 5,496,346; 5,514,154; 5,569,295; 5,603,721; 5,636,641; 5,649,952; 5,728,158; 5,735,893; 5,759,192; 5,780,807; 5,868,706; 6,056,776; 6,131,266; 6,179,810; 6,273,911; 6,309,412; 6,312,459; 6,369,355; 6,419,693; 6,432,133; 6,482,166; 6,485,511; 6,629,991; 6,629,994; 6,651,478; 6,656,220; 6,736,843; 6,746,423; 6,753,071; 6,818,247; 6,827,734; 6,887,219; 6,887,510; 6,890,318; 6,908,479; 6,921,411; 6,929,657; 6,939,373; 6,957,152, 5,716,981; 5,922,021, 6,120,536 the entirety of the disclosures of each are incorporated herein by reference thereto.

In one aspect, the methods and systems of the disclosed subject matter can lower matrix metalloproteinase (“MMP”) activity. MMP's are zinc-dependent proteases. MMPs have an important role in tissue remodeling associated with various physiological and pathological processes such as morphogenesis, angiogenesis, and tissue repair. Recent data suggests an active role of MMPs in the pathogenesis of Aortic Aneurysm. Excess MMPs degrade the structural proteins of the aortic wall.

MMPs are inhibited by specific endogenous tissue inhibitor of metalloproteinases (TIMPs), which comprise a family of four protease inhibitors: TIMP-1, TIMP-2, TIMP-3, and TIMP-4. Synthetic inhibitors generally contain a chelating group that binds the catalytic zinc atom at the MMP active site tightly. Common chelating groups include hydroxamates, carboxylates, thiols, and phosphinyls. Hydroxymates are particularly potent inhibitors of MMPs and other zinc-dependent enzymes, due to their bidentate chelation of the zinc atom.

The inhibition of some MMPs, such as MMP14 and TGF-β, can cause blood vessels to become leaky and enhance delivery of the therapeutic agent to the infracted tissue. Accordingly, the zinc formulation described can promote greater efficacy of a therapeutic agent if delivered with a therapeutic agent concurrently or otherwise. Various therapeutic agents can be included in the zinc chelator formulation. Suitable therapeutic agents include: paclitaxel, docetaxel, rapamycin, everolimus, zotarolimus, and any combination thereof. In some embodiments, the biologic is a protein or combination of multiple proteins such as, but not limited to, vascular endothelial growth factor (VEGF), basic fibroblast growth factor (bFGF), acidic fibroblast growth factor (aFGF), platelet-derived growth factor (PDGF), platelet-derived endothelial growth factor (PDEGF), placental derived growth factor, angiopoietin-1 (Ang-1), angiopoietin-2 (Ang-2), insulin-like growth factor 1 (IGF-1), insulin-like growth factor-2 (IGF-2), muscle derived insulin-like growth factor (mIGF), transforming growth factor-alpha (TGF-α), transforming growth factor-beta (TGF-β), hepatocyte growth factor (HGF), stem cell factor (SCF), hematopoietic growth factor or granulocyte colony-stimulating factors (G-CSF), granulocyte macrophage colony-stimulating factors (GM-CSF), nerve growth factor (NGF), growth differentiation factor-9 (GDF9), epidermal growth factor (EGF), stromal derived growth factor-1α (SDF-1α) neurotrophins, erythropoietin (EPO), thrombopoieten (TPO), myostatin (GDF-8), leukemia inhibitory factor (LIF), tumor necrosis factor-alpha (TNF-α), sonic hedgehog protein (Shh). Additionally, stem cells may be used.

The zinc chelator formulation described herein can inhibit the zinc from participating in MMP activity, thereby lowering the MMP activity. Lowering MMP activity results in preventing excessive breakdown or tissue and is helpful in tissue remodeling. As stated above MMP degradation of extracellular matrix is believed to occur and thin the infracted heart wall. The thinner wall then results in increased wall stress and heart failure. By reducing MMP activity will slow the thinning of the LV wall.

In one embodiment, the formulation is included in a coating applied to the medical device. The coating can be biodegradable. In accordance with one embodiment, the coating composition can include a solvent and a polymer dissolved in the solvent and optionally a wetting fluid. Representative examples of polymers that may be used as a coating for an implantable medical device includes, but is not limited to, poly(N-acetylglucosamine) (Chitin), Chitosan, poly(3-hydroxyvalerate), poly(lactide-co-glycolide), poly(3-hydroxybutyrate), poly(4-hydroxybutyrate), poly(3-hydroxybutyrate-co-3-hydroxyvalerate), polyorthoester, polyanhydride, poly(glycolic acid), poly(glycolide), poly(L-lactic acid), poly(L-lactide), poly(D,L-lactic acid), poly(D,L-lactide), poly(L-lactide-co-D,L-lactide), poly(caprolactone), poly(L-lactide-co-caprolactone), poly(D,L-lactide-co-caprolactone), poly(glycolide-co-caprolactone), poly(trimethylene carbonate), polyester amide, poly(glycolic acid-co-trimethylene carbonate), co-poly(ether-esters) (e.g. PEO/PLA), polyphosphazenes, biomolecules (such as fibrin, fibrinogen, cellulose, starch, collagen and hyaluronic acid), polyurethanes, silicones, polyesters, polyolefins, polyisobutylene and ethylene-alphaolefin copolymers, acrylic polymers and copolymers, vinyl halide polymers and copolymers (such as polyvinyl chloride), polyvinyl ethers (such as polyvinyl methyl ether), polyvinylidene halides (such as polyvinylidene chloride), polyacrylonitrile, polyvinyl ketones, polyvinyl aromatics (such as polystyrene), polyvinyl esters (such as polyvinyl acetate), acrylonitrile-styrene copolymers, ABS resins, polyamides (such as Nylon 66 and polycaprolactam), polycarbonates, polyoxymethylenes, polyimides, polyethers, polyurethanes, rayon, rayon-triacetate, cellulose acetate, cellulose butyrate, cellulose acetate butyrate, cellophane, cellulose nitrate, cellulose propionate, cellulose ethers, and carboxymethyl cellulose. Additional representative examples of polymers that may be especially well suited for use in fabricating embodiments of implantable medical devices disclosed herein include ethylene vinyl alcohol copolymer (commonly known by the generic name EVOH or by the trade name EVAL), poly(butyl methacrylate), poly(vinylidene fluoride-co-hexafluoropropene) (e.g., SOLEF 21508, available from Solvay Solexis PVDF, Thorofare, N.J.), polyvinylidene fluoride (otherwise known as KYNAR, available from ATOFINA Chemicals, Philadelphia, Pa., or Kynar 2750, available from Arkema), ethylene-vinyl acetate copolymers, poly(vinyl acetate), styrene-isobutylene-styrene triblock copolymers, and polyethylene glycol.

“Solvent” is defined as a liquid substance or composition that is compatible with the polymer and is capable of dissolving the polymer at the concentration desired in the composition. Examples of solvents include, but are not limited to, dimethylsulfoxide (DMSO), chloroform, acetone, water (buffered saline), xylene, methanol, ethanol, 1-propanol, tetrahydrofuran, 1-butanone, dimethylformamide, dimethylacetamide, cyclohexanone, ethyl acetate, methylethylketone, propylene glycol monomethylether, isopropanol, isopropanol admixed with water, N-methylpyrrolidinone, toluene, and combinations thereof.

In yet another aspect of the subject matter, a method of modulating an infarct tissue is provided. The method includes administering a formulation comprising zinc chelator and a vasodilator to the coronary vasculature. The formulation by lowering the MMPs modulates the infracted area of the tissue. In one embodiment, the method includes local delivery of the formulation to the injury site. The formulation comprising the zinc chelator is applies directly to the by contact in some embodiments. For example, the local delivery can be achieved via a balloon catheter, stent, or stent graft. Additionally, delivery can be delivered via intracoronary injection or infusion, intramyocardial needle injection, via open heart surgery and needle injection to the heart. Additionally it may be provided by a coating on a stent or scaffold, or balloon, or other device.

It is understood that the subject matter described herein is not limited to particular embodiments described, as such may, of course, vary. It is also understood that the terminology used herein is for the purpose of describing particular embodiments only, and is not intended to be limiting, since the scope of the present subject matter is limited only by the appended claims. Where a range of values is provided, it is understood that each intervening value between the upper and lower limit of that range and any other stated or intervening value in that stated range, is encompassed within the disclosed subject matter.

What is claimed is:

1. A formulation comprising:
a zinc chelator,
a biomaterial, and
a transmural transport enhancer, and optionally a therapeutic agent.

2. The formulation of claim 1, wherein the zinc chelator is bound to an agent.

3. The formulation of claim 2, wherein the agent is selected from the group comprising acetohydroxamic acid, N-(methyl)mercaptoacetamide, 3-Hydroxy-pyran-4-one, 1-Hydroxy-1H-pyridin-2-one, 3-Hydroxy-1-methyl-1H-pyridin-2-one, 3-Hydroxy-2-methyl-pyridin-4-one, 3-Hydroxy-1,2-dimethyl-1H-pyridin-4-one, 1-Hydroxy-1H-pyridine-2-thione, 3-Hydroxy-2-methyl-pyran-4-thione, 3-Hydroxy-1H-pyridin-2-one, 3-Hydroxy-pyran-4-thione, 3-Hydroxy-1-methyl-1H-pyridine-2-thione, 3-Hydroxy-1,2-dimethyl-1H-pyridine-4-thione, and any combination thereof.

4. The formulation of claim 1, wherein the zinc chelator is bound to a polymer.

5. The formulation of claim 4, wherein the polymer comprises polyglutamic acid or polymers of polyglutamic acid.

6. The formulation of claim 1, wherein the zinc chelator is bound to the biomaterial.

7. The formulation of claim 6, wherein the biomaterial comprises alginate.

8. The formulation of claim 7, wherein the biomaterial is alginate-EDTA copolymer.

9. The formulation of claim 1, wherein the transmural transport enhancer is a vasodilator.

10. The formulation of claim 9, wherein the vasodilator is ethanol.

11. The formulation of claim 9, wherein the vasodilator is an NO inducer.

12. The formulation of claim 11, wherein the NO inducer is at least one of sodium nitroprusside, nitroglycerin, sildenafil, Tadalafil, or PETN.

13. The formulation of claim 9, wherein the vasodilator includes at least one of tetrahydrocannabinol, atrial natriuretic peptide, L-arginine, NO, hydalazine, alpha blockers, ACE inhibitors or ARBs.

14. The formulation of claim 1, further comprising at least one of a poloxamer, pluronic or block copolymer.

15. An endovascular medical device comprising:
a formulation including a zinc chelator, a biomaterial and a vasodilator, the formulation disposed on the outer surface of the endoluminal medical device.

16. The endovascular medical device of claim 15, wherein the formulation is incorporated into a coating applied to the outer surface of the endoluminal medical device.

17. The endovascular medical device of claim 15, wherein the coating is biodegradable.

18. The endovascular medical device of claim 15, wherein the zinc chelator is bound to an agent selected from the group consisting of: acetohydroxamic acid, N-(methyl)mercaptoacetamide, 3-Hydroxy-pyran-4-one, 1-Hydroxy-1H-pyridin-2-one, 3-Hydroxy-1-methyl-1H-pyridin-2-one, 3-Hydroxy-2-methyl-pyridin-4-one, 3-Hydroxy-1,2-dimethyl-1H-pyridin-4-one, 1-Hydroxy-1H-pyridine-2-thione, 3-Hydroxy-2-methyl-pyran-4-thione, 3-Hydroxy-1H-pyridin-2-one, 3-Hydroxy-pyran-4-thione, 3-Hydroxy-1-methyl-1H-pyridine-2-thione, 3-Hydroxy-1,2-dimethyl-1H-pyridine-4-thione, and any combination thereof.

19. The endovascular medical device of claim 15, wherein the zinc chelator is bound to a polymer.

20. The endovascular medical device of claim 19, wherein the polymer comprises polyglutamic acid or polymers of polyglutamic acid.

21. The endovascular medical device of claim 15, wherein the zinc chelator is bound to the biomaterial.

22. The endovascular medical device of claim 21, wherein the biomaterial comprises alginate.

23. The endovascular medical device of claim 22, wherein the biomaterial is alginate-EDTA copolymer

24. The endovascular medical device of claim 15, wherein the vasodilator is an NO inducer.

25. The endovascular medical device of claim 24, wherein the NO inducer is sodium nitroprusside, nitroglycerin, sildenafil, Tadalafil, or PETN.

26. The endovascular medical device of claim 15, wherein the vasodilator is ethanol.

27. The endovascular medical device of claim 15 wherein the vasodilator includes at least one of tetrahydrocannabinol, atrial natriuretic peptide, L-arginine, NO, hydalazine, alpha blockers, ACE inhibitors or ARBs.

28. The endovascular medical device of claim 16, wherein the coating includes at least one of a poloxamer, pluronic or block copolymer.

29. The endovascular medical device of claim 15, wherein the medical device is a stent or a stent graft.

30. The endovascular medical device of claim 15, wherein the medical device is a balloon.

31. A method of modulating an infarct, the method comprising:
administering a formulation including a zinc chelator and a vasodilator to the coronary vasculature, wherein the formulation modulates an infracted area of a tissue after an ischemic event.

32. The method of claim 31, wherein the formulation includes a biomaterial.

33. The method of claim 31, wherein the zinc chelator is a pendant group to a polymer.

34. The method of claim 31, wherein the zinc chelator is a pendant group to a biomaterial.

35. The method of claim 31, wherein the vasodilator is selected from the group consisting of ethanol, NO inducers such as sodium nitroprusside, nitroglycerin, sildenafil, Tadalafil, PETN, tetrahydrocannabinol, atrial natriuretic peptide, L-arginine, NO, hydalazine, alpha blockers, ACE inhibitors and ARBs.

36. The method of claim 31, wherein the formulation is N,N,N,N-tetrakis(2-pyridlmethyl)ethylenediamine, alginate or alginate-EDTA copolymers and nitroprusside.

37. The method of claim 31, wherein the formulation is N,N,N,N-tetrakis(2-pyridlmethyl)ethylenediamine, conjugated polyglutamic acid or copolymer of poly glutamic acid, alginate or alginate EDTA copolymers and nitroprusside.

38. The method of claim 31, wherein the formulation is N,N,N,N-tetrakis(2-pyridlmethyl)ethylenediamine, conjugated alginate or alginate-EDTA copolymers, alginate or alginate EDTA copolymers and nitroprusside.

39. The method of claim 31, wherein the formulation comprises a zinc binding agent.

40. The method of claim 39, wherein the binding agent is selected from the group comprising acetohydroxamic acid, N-(methyl)mercaptoacetamide, 3-Hydroxy-pyran-4-one, 1-Hydroxy-1H-pyridin-2-one, 3-Hydroxy-1-methyl-1H-pyridin-2-one, 3-Hydroxy-2-methyl-pyridin-4-one, 3-Hydroxy-1,2-dimethyl-1H-pyridin-4-one, 1-Hydroxy-1H-pyridine-2-thione, 3-Hydroxy-2-methyl-pyran-4-thione, 3-Hydroxy-1H-pyridin-2-one, 3-Hydroxy-pyran-4-thione, 3-Hydroxy-1-methyl-1H-pyridine-2-thione, and 3-Hydroxy-1,2-dimethyl-1H-pyridine-4-thione.
